(19) United States
(12) Patent Application Publication
(10) Pub. No.: US 2023/0240749 A1
(43) Pub. Date: Aug. 3, 2023

(54) SYSTEMS AND METHODS FOR CONTROLLING SURGICAL TOOLS BASED ON BONE DENSITY ESTIMATION

(71) Applicant: Mazor Robotics Ltd., Caesarea (IL)

(72) Inventors: Amir Lev-Tov, Tel Aviv (IL); Itay Ben Shushan, Rehovot (IL); Eliyahu Zehavi, Tel Aviv (IL)

(21) Appl. No.: 17/590,025

(22) Filed: Feb. 1, 2022

(51) Int. Cl. A61B 34/10; A61B 34/32; A61B 34/00; A61B 5/00; A61B 90/00

(52) U.S. Cl. CPC A61B 34/10; A61B 34/32; A61B 34/25; A61B 5/4509; A61B 5/4566; A61B 90/37; A61B 2090/3762; A61B 2034/105

(57) ABSTRACT

A method of estimating bone mineral density according to at least one embodiment of the present disclosure includes receiving one or more images of an anatomical element; generating, based on the one or more images of the anatomical element, a three-dimensional mask for the anatomical element; generating, based on the three-dimensional mask for the anatomical element, a transformed three-dimensional mask for at least a portion of the anatomical element; filtering the one or more images of the anatomical element with the transformed three-dimensional mask for the at least a portion of the anatomical element; and determining, based on the filtering, a bone mineral density for the at least a portion of the anatomical element.

### BACKGROUND

The present disclosure is generally directed to robotic surgery, and relates more particularly to robotic surgery based on image information.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A method of estimating bone mineral density according to at least one embodiment of the present disclosure comprises: receiving one or more images of an anatomical element; generating, based on the one or more images of the anatomical element, a three-dimensional mask for the anatomical element; generating, based on the three-dimensional mask for the anatomical element, a transformed three-dimensional mask for at least a portion of the anatomical element; filtering the one or more images of the anatomical element with the transformed three-dimensional mask for the at least a portion of the anatomical element; and determining, based on the filtering, a bone mineral density for the at least a portion of the anatomical element.

Any of the aspects herein, wherein the anatomical element comprises a vertebra, and wherein the at least a portion of the anatomical element comprises a sub-anatomic region of the vertebra.

Any of the aspects herein, wherein the sub-anatomic region comprises a cortical region.

Any of the aspects herein, wherein the sub-anatomic region comprises a trabecular region.

Any of the aspects herein, wherein the transformed three-dimensional mask for the anatomical element comprises at least one of a trabecular mesh representing a trabecula portion of the vertebra or a cortical mask representing a cortical portion of the vertebra.

Any of the aspects herein, wherein generating the transformed three-dimensional mask for the at least a portion of the anatomical element comprises: generating a trabecular mask for the anatomical element; and generating a cortical mask for the anatomical element.

Any of the aspects herein, further comprising: determining, for a trabecular portion of a vertebra and with the trabecular mask, the bone mineral density for the trabecular portion of the vertebra; and determining, for a cortical portion of the vertebra and with the cortical mask, the bone mineral density for the cortical portion of the vertebra.

Any of the aspects herein, wherein the bone mineral density is measured based on Hounsfield Units.

Any of the aspects herein, further comprising: determining an average bone mineral density for a plurality of sub-volumes of the anatomical element.

Any of the aspects herein, further comprising: providing information describing the bone mineral density for the at least a portion of the anatomical element to a surgical robot as part of a surgical plan.

A method of operating a surgical robot according to at least one embodiment of the present disclosure comprises: receiving an estimate of bone mineral density for an anatomical element; initiating a surgical maneuver on the anatomical element with the surgical robot; receiving sensor information from a robotic sensor during the surgical maneuver; comparing the sensor information with an anticipated sensor reading, wherein the anticipated sensor reading is determined based on the estimate of bone mineral density for the anatomical element; and controlling the surgical robot during the surgical maneuver based on the comparison of the sensor information with the anticipated sensor reading.

Any of the aspects herein, wherein the surgical maneuver comprises at least one of drilling, milling, and cutting the anatomical element.

Any of the aspects herein, wherein the estimate of bone mineral density for the anatomical element is related to a particular volume of the anatomical element.

Any of the aspects herein, wherein the sensor information comprises at least one of drilling resistance, drilling torque, drilling depth, and force on an arm of the surgical robot.

Any of the aspects herein, wherein controlling the surgical robot comprises automatically stopping the surgical maneuver.

Any of the aspects herein, wherein controlling the surgical robot comprises allowing the surgical maneuver to continue in an autonomous fashion.

Any of the aspects herein, wherein the anatomical element comprises a vertebra.

Any of the aspects herein, wherein the estimate of bone mineral density for the anatomical element is determined with a CT scan or x-ray of the anatomical element.

Any of the aspects herein, wherein the estimate of bone mineral density for the anatomical element is determined preoperatively as a DXA t-score.

A system according to at least one embodiment of the present disclosure comprises: a first sensor; a surgical tool; a processor; and a memory including data stored thereon that, when processed by the processor, cause the processor to: receive an estimate of bone mineral density for an anatomical element; initiate a surgical maneuver on the anatomical element with the surgical tool; receive information from the first sensor during the surgical maneuver; compare the information with an anticipated sensor reading, wherein the anticipated sensor reading is determined based on the estimate of bone mineral density for the anatomical element; and control the surgical tool during the surgical maneuver based on the comparison of the information with the anticipated sensor reading.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present invention will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is an image of an anatomical element according to at least one embodiment of the present disclosure;

FIG. 2B is a segmentation mask of the anatomical element according to at least one embodiment of the present disclosure;

FIG. 2C is a masked image of the anatomical element according to at least one embodiment of the present disclosure;

FIG. 2D is a three-dimensional (3D) rendering of the anatomical element according to at least one embodiment of the present disclosure;

FIG. 2E is an estimated bone density of the anatomical element according to at least one embodiment of the present disclosure;

FIG. 3 is a flowchart according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple All, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Robotic surgical systems may use closed loop control systems that function based on clinical parameters such as bone thickness. For example, the robot may be configured to drill, or perform any other function, to a specific distance or depth based on the bone thickness.

Performing robotic drilling, milling, cutting, or any other operation in an automatic manner in any volume inside a bone (e.g., a vertebra) may be accomplished by the embodiments disclosed herein. For instance, embodiments of the present disclosure may determine or estimate Bone Mineral Density (BMD) information and convert such information into bone thickness in various locations of the anatomical element(s) into which the robot drills, mills, cuts, etc. One method of determining bone thickness includes segmenting the bone (e.g., a vertebra) into a cortical portion (e.g., a harder outer portion of the bone) and a trabecular part (e.g., a softer inner portion of the bone). Such information may be processed (e.g., by a computing device, processor, or other processing component) and used to control the automatic drilling, milling, cutting, etc. of the robot. Additionally or alternatively, the information may be used to facilitate control of any semi-autonomous processes and/or may be provided as feedback to the surgeon (e.g., by providing a notification to the surgeon that the robot is within the cortical or the trabecular part of the anatomical element).

In some embodiments, the robot may include drilling, milling, cutting, etc. components (e.g., different surgical tools) attached to an end effector of the robot. The surgical tools may be controlled, at least in part, based on the hardness/BMD information (e.g., the drill speed, torque parameters, etc. of a surgical tool may be set, controlled, and/or monitored by a surgical system) while the surgical tool is being operated (e.g., autonomously operated, semi-autonomously operated, etc.). In some embodiments, the surgical system or components thereof (e.g., a processor) may monitor real-time a correlation between the torque of the surgical tool and a predicted torque (e.g., a torque predicted based on the BMD information). Such monitoring may be measured, for example, using one or more sensors disposed proximate the surgical tool. When the measured torque and the predicted torque are different, the system may generate an alert signaling a mismatch. Such a mismatch may also disable or stop the surgical tool to facilitate surgical safety.

In some embodiments, the estimation of the BMD may commence with capturing or receiving Computed Tomography (CT) scans, 3D X-Ray reconstructions, and/or other images of one or more anatomical elements, such as anatomical elements subject to an operation, surgery, or surgical procedure (e.g., one or more vertebrae). The scans may be segmented (e.g., using segmentation and/or image processing) to create 3D masks for each anatomical element in the scans (e.g., each vertebra present in the CT scan). A mask (e.g., a vertebra mask) may be used to filter the CT scan and calculate a general BMD estimation (based on, for example, Hounsfield Units (HU)) for each anatomical element. Hounsfield units are dimensionless units universally used in CT scanning to express CT numbers in a standardized and convenient form. Hounsfield units are obtained from a linear transformation of measured attenuation coefficients. The transformations are based on the arbitrarily-assigned densites of air and pure water. For example, the radiodensity of distilled water at a standard temperature and pressure (STP) of zero degrees Celcius and 105 pascals is 0 HU; the radiodensity of air at STP is −1000 HU.

The mask may be converted into a 3D mesh and a mesh smoothing may be applied. After smoothing, one or more faces normal to the mesh may be determined. Based on the normal faces, a cylindrical shape may be used to sample the CT scans. A cortical width of the anatomical element may be determined from the sampled data (e.g., using thresholding, full width half max, gradient approach, etc.). From the sampled width, the cortical width of the anatomical element at every point of the anatomical element may be estimated (e.g., using interpolation).

Using the measured cortical widths at each point on the anatomical element, a 3D trabecular mesh may be generated that represents the trabecular portion of the vertebra. The 3D trabecular mesh may be converted into a standard 3D mask, and the opposite of the 3D mask (e.g., the cortical mask) may be determined.

Using both the cortical and the trabecular masks, the depiction of the vertebra may be segmented into cortical and trabecular parts. The CT scans may be filtered with the cortical and trabecular masks and a BMD estimation (based on HU) may be estimated. In some embodiments, a Magnetic Resonance Image (MRI) bone segmentation may be used instead of using an X-Ray based volumetric image. In some embodiments, the a sub-anatomical vertebra segmentation is used. In such embodiments, the above-mentioned computations/determinations may be applied, and corresponding parameters are determined for each sub-anatomical segment of the vertebra.

The surgical tool (e.g., a surgical drill), may be operated and/or controlled by a controller, processor, or other computing element (e.g., a computing device) and may include a feedback loop. The feedback may be generated based on received and/or processed information from one or more sensors (e.g., hardness sensors, resistance sensors, force sensors, torque sensors, etc.). In some embodiments, the controller may take into account the widths/depths of different bone layers (e.g., cortical and trabecular) in the bone in a given trajectory through which the bone is to be drilled. Additionally or alternatively, the distribution of the BMD estimation of each 3D cylinder (or other shape) representing a volume into which the surgical tool will drill may be provided to the controller to control the drill.

The feedback loop may use, for example, a PID (Proportional-Integral-Derivative) controller and/or Artificial Intelligence (AI) models to determine parameter values for the surgical tool (e.g., drill speed, torque, etc.). The use of the feedback loop in combination with BMD estimation may beneficially enable greater estimation of when to stop drilling (to address issues of late continuation where the drilling extends too far into the bone) and/or when to continue drilling (to address early stopping issues where the drilling does not sufficiently drill into the bone). The feedback loop also beneficially enables the parameters of the drill (e.g., drill speed, power/torque, etc.) to be adjusted along with the changing densities along the drilling volume of the bone, allowing the drill to adjust not only when the bone switches from cortical to trabecular material, but rather to any type of density encountered during the drilling. Furthermore, the monitoring decreases the likelihood of drilling into the bone with a predicted torque that is inappropriate for the given bone density (e.g., the torque is too low to drill through the bone, the torque is too high resulting in unnecessary damage to the bone or the patient, etc.). In some embodiments, the surgical tool may be calibrated based on the BMD estimation. In other words, the torque, drill speed, and/or other tool parameters may be selected based on the BMD estimation (e.g., greater density may result in a higher drill torque).

The use of the above-mentioned surgical tool and BMD estimation is not particularly limited to vertebrae, and the use of the aspects of the present disclosure may be directed toward any anatomical element and toward any general use of a surgical tool to operate on bone. For instance, the aspects may be used to drill/mill a vertebra body (e.g., entering through cortical bone, then through trabecular bone, then again through cortical bone to drill through the entire body); perform one or more lamina cuts that avoid damaging the dura; drilling/milling/cutting while avoiding violating 3D pedicle boarders; determining end plate width information to insert vertebral cages; combinations thereof; and/or the like.

In some embodiments, a surgical system may perform a BMD estimation, and then tune the robotic surgical tool based on the BMD estimation. The BMD estimation may use a 3D scan of the spine (e.g., a CT scan) to estimate the BMD. The 3D scan may be segmented into cortical and trabecular parts of the bone, and the cortical width at every point of the bone (or at every point relevant to the surgery or surgical procedure) may be estimated.

Once the BMD estimation is established, a surgical tool may be operated based on the BMD estimation using, for example, a feedback loop. The set tool parameters may be compared with measured tool parameters based on one or more sensors. When the set tool parameters fail to match the measured tool parameters within a degree of certainty, the operation of the surgical tool may be stopped as a safety precaution. In some embodiments, the BMD estimation may occur at sub-anatomical levels (e.g., regions within each of the cortical and/or trabecular portions)

The use of the above-mentioned aspects may beneficially enable surgical operation on bone based on determined characteristics such as hardness and/or thickness at any sub-anatomic location of the bone (e.g., the spine or one or more vertebrae thereof). While some embodiments discussed herein are directed toward the autonomous operation of a surgical tool, the estimation of the BMD may beneficially enable more detailed surgical plans to be constructed and/or beneficially enable a surgeon or other physician to make surgical decisions based on the BMD.

Embodiments of the present disclosure provide technical solutions to one or more of the problems of (1) operating on bone with inappropriate surgical tool parameters, (2) drilling too far or not far enough into a bone, and (3) exposing a patient to unnecessary radiation during operation of the surgical tool.

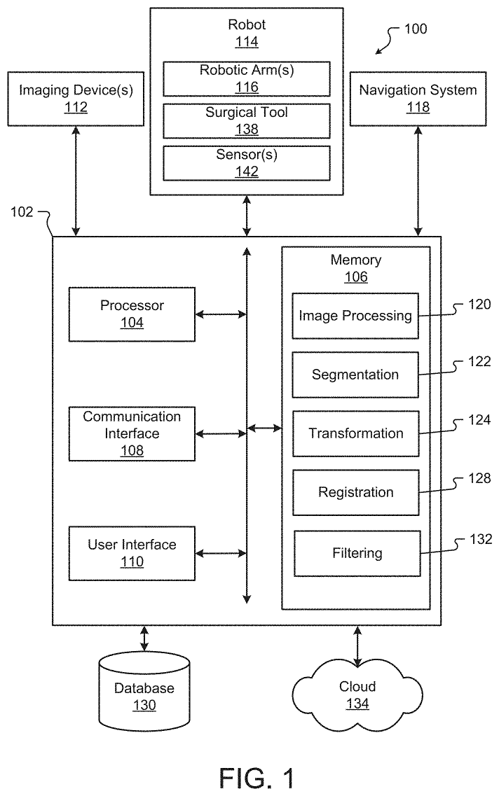

Turning first to FIG. 1, a block diagram of a system 100 according to at least one embodiment of the present disclosure is shown. The system 100 may be used to estimate the BMD of a bone (e.g., a vertebra); to operate a surgical tool (e.g., a surgical drill, a surgical saw, etc.) with one or more operating parameters (e.g., drill speed, torque, etc.) chosen based on the estimated BMD; to measure the operating parameters of the drill and compare the measured operating parameters to predicted operating parameters; to shut off or stop the surgical tool when the measured parameters and the predicted parameters are not within a required degree of certainty; to control, pose, and/or otherwise manipulate a surgical mount system, a surgical arm, and/or surgical tools attached thereto; and/or to carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 300 and/or 400 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, registration 128, and/or filtering 132. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The system 100 may comprise a surgical tool 138. The surgical tool 138 may be configured to drill, mill, cut, saw, ream, tap, etc. into patient anatomy (e.g., soft tissues, bone, etc.). In some embodiments, the system 100 may comprise multiple surgical tools, with each surgical tool performing a different surgical task (e.g., a surgical drill for drilling, a surgical mill for milling, etc.). In other embodiments, the surgical tool 138 may provide multiple different types of surgical maneuvers (e.g., the surgical tool 138 may be able to receive one or more different tool bits, such that the surgical tool 138 can drill, mill, cut, saw, ream, tap, etc. depending on the tool bit coupled with the surgical tool 138). The surgical tool 138 may be operated autonomously or semi-autonomously. In some embodiments, the surgical tool 138 may be attached to a robotic arm 116, such that movement of the robotic arm 116 correspondingly causes movement in the surgical tool 138. In other words, the surgical tool 138 may be gripped, held, or otherwise coupled to and controlled by the robotic arm 116. As such, the pose (e.g., position and orientation) of the surgical tool 138 may be controlled by the pose of the robotic arm 116.

The surgical tool 138 can be controlled by one or more components of the system 100, such as the computing device 102. In some embodiments, the computing device 102 may be capable of receiving or retrieving data or other information (e.g., from the database 130, from one or more sensors 142, from the imaging devices 112, etc.), process the information, and control the surgical tool 138 based on the processed information. For example, the computing device 102 may receive or process information related to a BMD estimation, and adjust the operating parameters (e.g., drill speed, drill torque, etc.) and/or the pose of the surgical tool 138 based thereon. In some embodiments, the computing device 102 may implement a feedback loop, in conjunction with the one or more sensors 142, to adjust the surgical tool 138. The feedback loop may incorporate sensor data from the one or more sensors 142 and update the operating parameters and/or the pose of the surgical tool 138 accordingly.

The one or more sensors 142 may comprise force sensors, torque sensors, heat sensors, combinations thereof, and/or the like. The one or more sensors 142 may be disposed on or proximate to one or more of the robot 114, the robotic arm 116, the surgical tool 138, combinations thereof, and/or the like. The one or more sensors 142 may capture one or more measurements and send the measurements to the computing device 102. The one or more sensors 142 may be wired to or wirelessly connected to one or more components of the system 100 (e.g., in wireless communication with the computing device 102 and/or the processor 104).

In some embodiments, the one or more sensors 142 may measure data associated with the operation of the surgical tool 138. For instance, the one or more sensors 142 may comprise force and/or torque sensors capable of measuring forces on and/or the torque components connected thereto or disposed proximate thereto. In one embodiment, the one or more sensors 142 may measure the torque of the surgical tool 138 as the surgical tool 138 operates and send such measurements to the computing device 102, the processor 104, and/or other components of the system 100. The one or more sensors 142 may additionally or alternatively comprise heat sensors that may measure heat generated during the operation of the surgical tool 138 and send such measurements to the computing device 102, the processor 104, and/or other components of the system 100.

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 300 and/or 400 described herein. The system 100 or similar systems may also be used for other purposes.

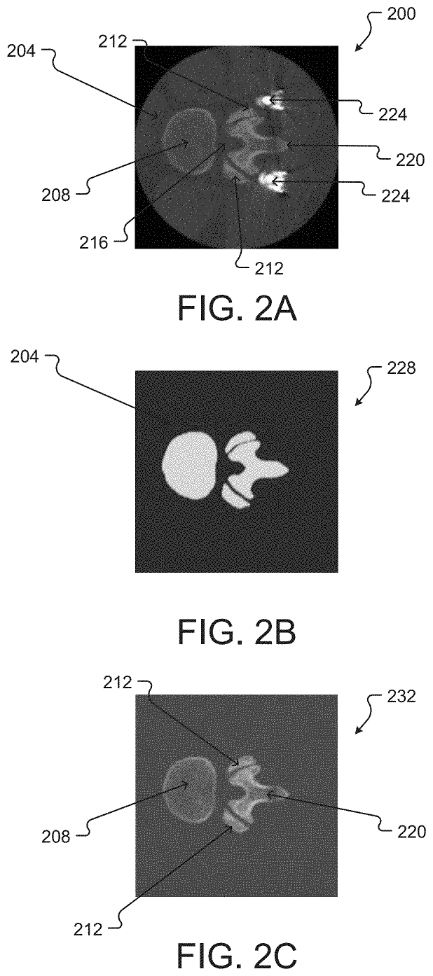

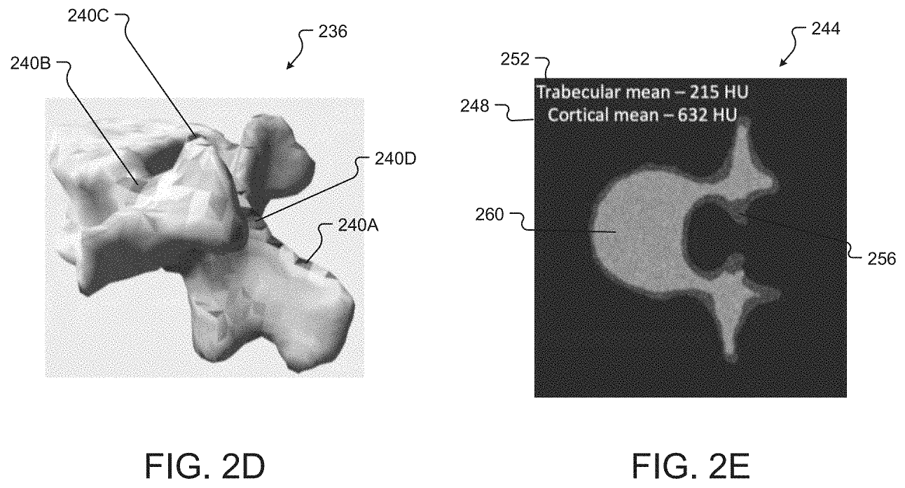

Turning to FIGS. 2A-2E, aspects for estimating a BMD of an anatomical element are shown in accordance with at least one embodiment of the present disclosure. FIG. 2A illustrates a scan 200 depicting an anatomical element 204. The scan 200 may be a CT scan or image, a 3D X-Ray scan (or a reconstruction and/or mapping of a plurality of 2D X-Ray scans into a 3D X-Ray scan), or the like that may be captured preoperatively (e.g., before the surgery or surgical procedure) or intraoperatively (e.g., during the surgery or surgical procedure). In some embodiments, the scan 200 may be captured intraoperatively and used to update a preoperative image, a surgical plan, combinations thereof, and/or the like.

The scan 200 may depict multiple anatomical elements (e.g., organs, bones, soft tissues, etc.). In one embodiment, the scan 200 may depict the spine or one or more vertebra thereof (e.g., a cervical vertebra, a thoracic vertebra, a lumbar vertebra, a sacrum, a coccyx vertebra, etc.). While the spine and vertebrae thereof are generally discussed herein, it is to be understood that other bones of a patient may be depicted in the scan 200 and/or subject to surgery or surgical procedures in accordance with the embodiments of the present disclosure. Non-limiting examples of anatomical element 204 depicted in the scan 200 include cranial bones, facial bones, upper and lower arm bones, hand bones, chest or thorax bones, bones in the pelvis, leg bones, and foot bones. As illustrated in FIG. 2A, the anatomical element 204 may be a vertebra comprising a body 208, one or more lamina 212, a spinal canal 216, a spinous process 220, and one or more artery canals 224.

The scan 200 may be processed to create a masked scan 232. The processing may include applying a segmentation mask 228 to the scan 200. The segmentation mask 228 may be a predetermined mask determined based on, for example, the type of scan, the position of the scan (e.g., the angle of the equipment used to capture the scan), the type of anatomy depicted in the scan, the type of surgery or surgical procedure, combinations thereof, and/or the like that can be used to segment the scan 200. In some embodiments, the segmentation mask 228 may be used by the segmentation 122 to segment the scan 200. In some embodiments, the segmentation mask 228 may be generated or determined using one or more machine learning models or artificial intelligence models that may take information such as the type of surgical procedure, the target anatomy (e.g., the type of vertebra to be drilled, milled, cut, etc.), or any other type of surgical information and output the segmentation mask 228. In some embodiments, the models used to generate the segmentation mask 228 may be trained on historical data for similar anatomical elements. For instance, if the scan 200 depicts a lumbar vertebra, the model that processes the scan 200 may be trained on images of other lumbar vertebrae to improve the segmentation of the lumbar vertebra from scan 200 to form the segmentation mask 228. Similarly, a scan 200 depicting a thoracic vertebra may implement models trained on images of thoracic vertebrae.

The segmentation mask 228 may then be applied to the scan 200 to create the masked scan 232. In some embodiments, the image processing 120 and/or the segmentation 122 may apply the segmentation mask 228 to the scan 200. For instance, the scan 200 and the segmentation mask 228 may be passed into one or more machine learning or artificial intelligence models that may process the scan 200 by applying the segmentation mask 228 to the scan 200. By applying the segmentation mask 228 to the scan 200, the resulting masked scan 232 may enable identification of the portions of the scan 200 depicting the vertebra. In some embodiments, the image processing 120 of the scan 200 using the segmentation mask 228 may include applying one or more filters (e.g., noise reduction filters, weighted average filters, etc.) to the scan 200 before and/or after applying the segmentation mask 228.

The resulting masked scan 232 may remove or omit elements of the scan 200. As shown in FIG. 2C, the application of the segmentation mask 228 to the scan 200 may result in the masked scan 232 depicting the body 208, the lamina 212, and the spinous process 220 of the anatomical element 204, while other anatomical features original present in the scan 200, such as the spinal canal 216 and the artery canals 224, may be removed or omitted (e.g., filtered out). In some embodiments, different anatomical features may be removed based on the segmentation mask 228. For instance, the segmentation mask 228 may cause superfluous or additional anatomical features unrelated to the anatomical element 204 (e.g., the scan may depict ribs and vertebrae, and the models are tuned to identify the vertebrae and remove the ribs when creating the segmentation mask 228) to be omitted or removed from the masked scan 232. In some embodiments, one or more segmentation masks may be applied to one or more anatomical elements (e.g., one or more vertebrae), such that multiple anatomical elements can be segmented based on the scan 200.

In some embodiments, the masked scan 232 may be converted into a 3D reconstruction 236. The conversion may include using one or more transformations 124 to the masked scan 232 to create the 3D reconstruction 236. In some embodiments, the transformation 124 may take the masked scan 232, as well as additional pose data, and generate the 3D reconstruction 236. Additionally or alternatively, segmentation 122 (e.g., using a Deep Neural Network, using filtering models, etc.) may sample one or more points (e.g., vertices) along the surface of the masked scan 232, generate a point cloud of the one or more points, and connect the points to form a plurality of faces for the 3D reconstruction 236. In some embodiments, additional mesh smoothing may be applied to the 3D reconstruction 236 (e.g., to remove outlying points, to provide a more uniform point density, etc.). In some embodiments, the 3D reconstruction 236 may be or comprise a point cloud (e.g., a set of datapoints in a 3D space).

One or more faces along the surface of the 3D reconstruction 236 may be used to estimate the width of the cortical portion of the anatomical element 204. The 3D reconstruction 236 may include faces 240A-240D along the surface thereof. The faces 240A-240D may be used to determine faces normal to the 3D reconstruction 236. For instance, the transformation 124 may receive the 3D reconstruction 236 and use the faces 240A-240D to determine directions normal to the faces 240A-240D. In some embodiments, the 3D reconstruction 236 may be sampled with a cylindrical shape (e.g., a rectangular cylinder) in the directions normal to each of the faces 240A-240D. For instance, the cylindrical shape may be placed normal to the face 240A, such that the cylindrical shape encompasses points of the point cloud forming the surface of the 3D reconstruction 236 as well as points under the surface of the 3D reconstruction 236. The sampling may include determining the relative density of points inside the cylindrical shape for each of the sampling locations (e.g., each of the surfaces of the 3D reconstruction 236). The collected densities may be processed (e.g., using filtering 132) to estimate the width of the cortical portion of the anatomical element 204. For instance, the filtering 132 may take the density values for each surface of the 3D reconstruction 236 and process the values (e.g., using thresholding, using full width half max, gradient descent methods, etc.) to estimate the width of the cortical portion of the anatomical element 204.

After the cortical width has been estimated, the widths may be further used to generate a trabecular mesh representing the trabecular portion of the vertebra. For example, filtering 132 may be applied to the 3D reconstruction 236 to remove the data associated with the estimated cortical width, leaving data representing the trabecular portion. Additionally, image processing 120 may be applied to the trabecular mesh to filter or manipulate the data associated with the trabecular portion (e.g., applying filters based on models of the trabecular portion of the anatomical element) to create a trabecular mask.

After the cortical and trabecular masks have been generated, both the cortical and trabecular masks may be applied to the scan 200. In some embodiments, one or more machine learning or artificial intelligence models may filter the scan 200 using the cortical and trabecular masks, such that the scan 200 is segmented into cortical and trabecular portions (e.g., such that the scan 200, when rendered to a display, show the cortical and trabecular portions in two different colors). In other words, the scan 200 may be filtered using the cortical and trabecular masks. Once filtered, image processing 120 may extract information (e.g., pixel information) to determine a BMD estimation 244. The extracted information may be processed by the image processing 120 to determine the Hounsfield Units (HU) associated with each of the cortical and trabecular portions of the anatomical element 204. In some embodiments, individual HU values may be associated with individual pixel values for the scan 200 (e.g., a CT scan). To generate an estimated HU, the image processing 120 may extract pixel information from each segment of the scan 200 that has been filtered with the cortical and trabecular masks. For instance, all pixels values within each segment (e.g., a cortical segment, a trabecular segment, etc.) may be extracted (e.g., using image processing 120) and averaged to generate an estimated HU of each segment. In other words, for the cortical segment, all pixel values residing therein may be averaged to determine an estimated HU for the cortical segment, while all pixel values within the trabecular segment may be separately averaged to determine an estimated HU for the trabecular segment.

In some embodiments, the BMD estimation 244 may be rendered to a display (e.g., a user interface 110), and may display a cortical value 248 (e.g., approximately 632 HU) and/or a trabecular value 252 (e.g., approximately 215 HU). Similarly, the rendering to the display may visually depict the segmented portions of the anatomical element 204, such as a cortical portion 256 displayed in a first color (e.g., blue) and a trabecular portion 260 displayed in a second color (e.g., green).

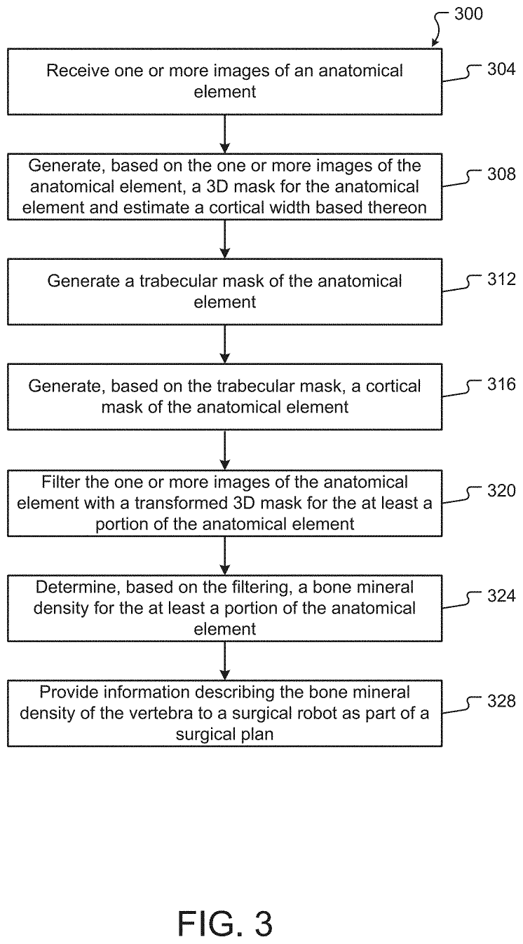

FIG. 3 depicts a method 300 that may be used, for example, to determine a BMD of an anatomical element based on one or more images of the anatomical element.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in the memory and executed by the processor may cause the processor to execute one or more steps of a function as shown in method 300. One or more portions of a method 300 may be performed by the processor executing any of the contents of memory, such as image processing 120, segmentation 122, transformation 124, registration 128, and/or filtering 132.

The method 300 comprises receiving one or more images of an anatomical element (step 304). The one or more images may be similar to or the same as the scan 200. Similarly, the anatomical element may be similar to or the same as anatomical element 204. In one embodiment, the anatomical element may be or comprise a vertebra of the spine. The one or more images may be received by, for example, a computing device 102, a processor 104, a navigation system 118, and/or one or more other components of the system 100. The one or more images may be captured preoperatively (e.g., a CT scan, a 3D X-Ray image, etc.). In some embodiments, the one or more images may be a plurality of 2D images (e.g., fluoroscopic images), which may be captured preoperative and/or intraoperatively. Images used may include combinations of different image types, such as CT images, 3D X-Ray image(s), and/or 2D fluorscopic images.

The method 300 also comprises generating, based on the one or more images of the anatomical element, a 3D mask for the anatomical element (step 308). The one or more images may be processed (e.g., using image processing 120) to generate a 3D mask. The 3D mask may be similar to or the same as the segmentation mask 228. In some embodiments, image processing 120 may take the one or more images as inputs (e.g., into a machine learning model, into an artificial intelligence model, etc.) and output the 3D mask. In some embodiments, the models used by the image processing 120 may be models trained on data associated with similar anatomical elements. For example, models trained on lumbar vertebrae may be used when the 3D mask generated from the one or more images is directed toward a lumbar vertebrae. In some embodiments, the one or more images may contain multiple anatomical elements, and a plurality of models may be able to separate each anatomical element into independent 3D masks. The 3D mask may then be converted into a 3D mesh (e.g., a point cloud or other 3D reconstruction of the vertebra such as 3D reconstruction 236) that depicts data associated with the vertebra.

The estimation of the cortical width may include determining one or more directions normal to the one or more locations on the surface of the 3D mesh. For instance, image processing 120 may use one or more models to determine normal directions for some or all of the locations (e.g., data points) of the 3D mesh. For one or more of the locations, the data may be sampled in the direction of the normal directions using a 3D shape (e.g., a cylinder). The image processing 120 may pass the 3D shape through the 3D mesh such that a collection of data points fall within the 3D shape. Each of the number of data points within the 3D shape may be used by the image processing 120 to determine a relative density of points for each location along the surface of the 3D mesh. Based on the density of points, filtering 132 may be used to determine a cortical width at each point of the surface of the 3D mesh. For instance, the filtering 132 may use thresholding, full width at half max, gradient approaches, combinations thereof, and/or the like to estimate the cortical width of the vertebra represented by the 3D mesh.

The method 300 also comprises generating a trabecular mask of the anatomical element (step 312). The trabecular mask may be generated by using the estimated cortical width. For instance, by subtracting or removing (e.g., using filter 132) the data points associated with the cortical width, a first set of outermost points in the 3D mesh may be removed, leaving a collection of “inner” data points that correspond to the trabecular portion of the vertebra. In some embodiments, mesh smoothing, or other filtering, may be performed on the trabecular portion data. The data in the mesh may be transformed into a trabecular mask. The transformation may be performed, for example, by one or more transformations 124 that receive the 3D mesh data and construct a trabecular mask therefrom. In some embodiments, the transformations 124 may be or comprise models trained on trabecular data such that the transformation of the data into the mask occurs with a certain degree of accuracy. In some embodiments, the models may have internal mechanisms (e.g., threshold values for accuracy) to ensure that the trabecular mask, when applied to a scan or other image, segments the trabecular portion of the vertebra to a certain degree of statistical certainty.

The method 300 also comprises generating, based on the trabecular mask, a cortical mask of the anatomical element (step 316). In some embodiments, the cortical mask may be generated by calculating the opposite of the trabecular mask. For instance, the data associated with the trabecular mask may be removed from the 3D mesh, leaving the data points associated with the cortical portion of the vertebra. The cortical data may then be passed into one or more transformations 124 that transform the cortical data into a cortical mask. In some embodiments, the transformation of the cortical data into the cortical mask may be similar to or the same as the transformation of the trabecular data into the trabecular mask.

The method 300 also comprises filtering the one or more images of the anatomical element with a transformed 3D mask for the at least a portion of the anatomical element (step 320). The step 320 may utilize or implement filtering 132 to segment the images of the anatomical element into cortical and trabecular portions. For example, the filtering 132 may comprise one or more machine learning and/or artificial intelligence models that receive the one or more images, the trabecular mask, and the cortical mask and output the anatomical element segmented into the trabecular portions and the cortical portions. In some embodiments, the anatomical element may be rendered to a display (e.g. user interface 110). In such embodiments, different segments of the anatomical element may be rendered or displayed in different colors (e.g., the cortical portion of the vertebra rendered in a first color and the trabecular portion of the vertebra rendered in a second color), which may enable a user (e.g., a surgeon, a member of a surgical staff, etc.) to review the accuracy of the segmenting. In some embodiments, an inaccurate segmenting (e.g., as determined by the user, as determined by threshold accuracy requirements built into the segmenting models, etc.) may result in new cortical and/or trabecular masks being generated, and/or different filtering processes being applied to the images of the anatomical element. Such changes to the masks or filtering processes may be repeated until a satisfactory segmentation of the anatomical element occurs (e.g., until the surgeon is content with the segmenting, until the accuracy thresholds of the models are met, etc.).

The method 300 also comprises determining, based on the filtering, a bone mineral density for the at least a portion of the anatomical element (step 324). Once filtered, the segmented portions of the anatomical element may be processed (e.g., using image processing 120) to determine the BMD of each segmented portion. In some embodiments, the processing may use machine learning and/or artificial intelligence models that receive the segmented portions of the anatomical element; the cortical width estimations; image data including pixel values, information associated with how the image was captured (e.g., energy used in the CT scan or X-Ray, distance between emitter and detector, etc.); the cortical and/or trabecular masks; combinations thereof; and/or the like and output an estimated BMD associated with each of the trabecular and cortical portions. In some embodiments, the models may be trained on data associated with similar scans, anatomical elements, and/or segmentations.

The method 300 also comprises providing information describing the bone mineral density of the vertebra to a surgical robot as part of a surgical plan (step 328). The BMD information may be incorporated, for example, into the surgical plan to tune the operating parameters of a surgical tool (e.g., a surgical drill). Additionally or alternatively, the BMD information may be incorporated into the surgery or surgical procedure, such that the computing device 102 (or any other component of the system 100) may monitor the operation of the surgical tool based at least in part on the BMD information. For example, the computing device 102 may monitor a surgical drill as the surgical drill drills into a vertebra. The computing device 102 may, based on the BMD information, know or be able to estimate the depth at which the drill passes through the cortical portion of the vertebra and into the trabecular portion (or vice versa), and may adjust the operating parameters of the surgical tool accordingly.

The present disclosure encompasses embodiments of the method 300 that comprise more or fewer steps than those described above, and/or one or more steps that are different than the steps described above.

Furthermore, it is to be noted that the BMD estimation discussed in the method 300 above, while describing cortical and trabecular portions of a vertebra, is not intended to be limited to such portions of a vertebra, and that the BMD of different segments, sub-volumes, or sub-anatomic elements of any bone may be assessed or estimated using the steps discussed in the method 300, as well as other steps of other methods discussed herein.

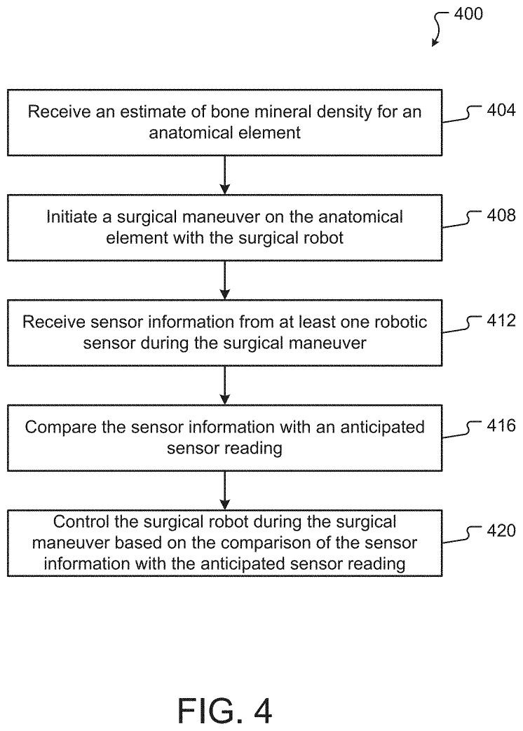

FIG. 4 depicts a method 400 that may be used, for example, to operate a surgical tool based on BMD.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as image processing 120, segmentation 122, transformation 124, registration 128, and/or filtering 132.

The method 400 comprises receiving an estimate of bone mineral density for an anatomical element (step 404). The estimated BMD may be an estimate (e.g., in HU) of the relative density of one or more portions of an anatomical element (e.g., anatomical element 204). In one embodiment, the estimated BMD may be of an anatomical element (e.g., a vertebra) subject to a surgery or surgical procedure. The received BMD may be received by, for example, a computing device 102, a processor 104, a navigation system 118, and/or any other component of the system 100.

In some embodiments, the estimated BMD may be based on, for example, data generated by applying one or more masks to one or more surgical images (e.g., a 3D CT scan, a 3D X-Ray reconstruction, etc.). In one embodiment, the estimated BMD may be generated based on one or more of the systems and/or methods described herein (e.g., using the method 300). Additionally or alternatively, the estimated BMD may be based on an estimate based on a preoperative bone density scan, such as a dual-energy x-ray absorptiometry (DXA). For instance, a t-score associated with the preoperative DXA may be used (e.g., by machine learning and/or artificial intelligence models) to estimate the BMD of the anatomical element.

The method 400 also comprises initiating a surgical maneuver on the anatomical element with the surgical robot (step 408). The surgical maneuver may include operating a surgical tool attached to, held or gripped by, or otherwise mechanically coupled to the surgical robot. For instance, the surgical robot may comprise a robotic arm (e.g., robotic arm 116) that holds a surgical tool (e.g., surgical tool 138), with the computing device 102 manipulating the pose of the robotic arm to change the pose of the surgical tool attached thereto. In some embodiments, the surgical robot and/or surgical tool may be similar to or the same as any respective surgical robot and/or surgical tool discussed herein. For instance, the surgical maneuver may be or comprise any one or more of drilling, milling, cutting, sawing, reaming, and/or tapping one or more portions of an anatomical element (e.g., bone or, more specifically, one or more portions of a vertebra).

In one embodiment, the surgical tool may comprise a surgical drill (e.g., a surgical tool capable of drilling one or more holes in a vertebra), which may be operated based on the BMD. The received BMD estimation may be used by the computing device 102 and/or the navigation system 118 to position the surgical drill. For instance, the BMD estimation may indicate that the cortical portion of the vertebra has a first hardness (e.g., a BMD of 620 HU) while the trabecular portion of the vertebra has a second hardness (e.g., a BMD of 200 HU). The difference in hardness values may require the surgical drill to operate at different parameters during the course of the drilling. As such, the computing device 102 may adjust the operating parameters of the surgical drill throughout the course of the surgery or surgical procedure. As an example, the surgical drill may operate with a first set of operating parameters (e.g., a first drill speed, a first torque, drilling at a first angle, etc.) while drilling through the cortical portion of the vertebra, and the computing device 102 may adjust the surgical drill to operate under a second set of operating parameters (e.g., a second drill speed, a second torque, drilling at a second angle, etc.). In some embodiments, only a portion of the operating parameters may change between the first set and the second set (e.g., the angle and drill speed may remain constant, but the drill torque parameter is changed) depending on, for example, the type of surgery or surgical procedure, sensor feedback, combinations thereof, and/or the like.

The method 400 also comprises receiving sensor information from at least one robotic sensor during the surgical maneuver (step 412). The robotic sensor may be or comprise one or more sensors (e.g., one or more sensors 142) disposed on and/or proximate to the surgical tool, patient anatomy, combinations thereof, and/or the like. The robotic sensors may, for example, measure operating parameters of the surgical drill (e.g., drill speed, torque, heat generated by the drilling, etc.) and send such measurements to the computing device 102 and/or the navigation system 118. In some embodiments, the robotic sensors may continually capture and transmit measurements to the computing device 102 and/or the navigation system 118. In some embodiments, the sensors may measure multiple different parameters associated with the operation of the surgical tool. Non-limiting examples include a drilling resistance (e.g., the amount of resistance the bone is providing); drilling torque; drilling depth (e.g., how far the surgical tool has drilled, milled, cut, etc. into the bone); one or more forces (e.g., forces on the robotic arm, forces on the surgical tool, forces on the bone, etc.); temperature of the surgical tool, bone surface, robotic arm or portions thereof; combinations thereof; and/or the like.

The method 400 also comprises comparing the sensor information with an anticipated sensor reading (step 416). The anticipated sensor reading may be based at least in part on the estimated BMD. One or more machine learning and/or artificial intelligence models may be implemented that receive the estimated BMD and the surgical tool operating parameters and generate anticipated sensor readings. For instance, the models may receive the current drilling depth of the surgical tool and the estimated BMD and output a predicted torque of the surgical tool that model anticipates will be measured by the sensors. The prediction may be then compared to the sensor readings, which may include a currently-measured torque. In some embodiments, the computing device 102 may compare multiple sensor readings. For instance, each of a predicted value and a measured value for torque, drill speed, heat generated by the drilling procedure, combinations thereof, and/or the like may be compared by the computing device 102 (e.g., using mathematical operations or a comparison model that determines a difference in value between the predicted value and the measured value).

In some embodiments, the difference between the anticipated sensor reading and the actual sensor reading may be compared to a threshold value (e.g., an integer, a percent, etc.). The threshold value may be a predetermined value, or a value determined based on the surgical plan, and may represent a tolerance of the system 100. Hence, a difference between the measured and anticipated operating parameter values that falls above the threshold value (or in some embodiments, below the threshold value) may indicate that the surgical drill is not operating within the safety requirements of the system 100. In other words, when the difference between how the surgical drill is operating and how the surgical drill should be operating (based on an estimated operating parameters) does not match within a certain degree of similarity, the computing device 102 may respond by changing the operation of the surgical tool, as discussed in step 420.

In cases where the difference is above (or below) a threshold value, the computing device 102 may generate an alert (e.g., a bell, an alarm, a flashing light, etc.) to indicate that the difference between the anticipated sensor value and the measured sensor value does not meet the standards or requirements of the system 100 (e.g., above/below the threshold value). In some embodiments, multiple differences may be compared against multiple different threshold values (e.g., differences between measured and anticipated torque and between measured and anticipated temperature of a surgical site may be compared against respective threshold values). In such embodiments, the alert may be generated when any one or more of the differences falls above (or below) the threshold value. As an example, the drill speed of the surgical tool may be within the drill speed threshold value (e.g., the surgical drill is operating at an appropriate speed as defined by the system 100), but the temperature of the drill may be too high (e.g., the temperature of the surgical tool is too high above the anticipated temperature value of the surgical drill such that the difference between the two is above a threshold temperature difference value). Since the temperature is too high, an alert may be generated by the computing device 102, and the operating parameters of the surgical drill and/or the surgical plan may be adjusted to lower the difference (e.g., less force is applied on the surgical drill in a direction of drilling, additional coolant is pumped into the surgical site, etc.).

The method 400 also comprises controlling the surgical robot during the surgical maneuver based on the comparison of the sensor information with the anticipated sensor reading (step 420). For instance, the parameters of the surgical drill (or other surgical tool) may be adjusted based on the comparison between the anticipated sensor reading and the measured sensors reading (e.g., the sensor reading created by the one or more sensors 142 positioned on or proximate the surgical drill, the patient anatomy, combinations thereof, and/or the like). When the comparison falls above (or below) a threshold value, the computing device 102 may determine that the surgical drill is operated differently than expected (e.g., the drill torque is too high, the drill speed is too low, etc.). In such cases, the computing device 102 may adjust the operation of the surgical drill to a different set of operating parameters (e.g., increase or decrease drill speed, increase or decrease torque, etc.) to reduce the difference between the anticipated sensor reading and the actual sensor reading. In some embodiments, the computing device 102 may use one or more control loops (e.g., PID controllers) or other control mechanisms (e.g., Kalman filtering, state space representations or other mathematical models, etc.) to adjust the surgical drill parameters such that the resulting output decreases the difference between the anticipated sensor reading and the actual sensor reading. Additionally or alternatively, the computing device 102 may use machine learning and/or artificial intelligence models trained on similar surgical procedures on similar anatomical elements to estimate the operating parameters of the surgical drill to reduce the difference.

In some embodiments, the adjustment may continue until the difference between anticipated and actual sensor readings meets the threshold level (e.g., falls above or below the threshold value, depending on how the difference is compared to the threshold). For instance, the initial comparison may indicate that the difference between the current drill speed (as measured by the sensors) and the anticipated drill speed (determined by a model predicting drill speed based on, for example, the BMD of the portion of the bone through which the surgical drill is drilling) falls above a threshold value (e.g., the surgical drill torque is too low, such that the surgical tool is not drilling through the bone). The computing device 102 may then cause the torque of the surgical tool to increase. This process may be continuously repeated (e.g., based on feeding the sensor measurements through a control/feedback system and adjusting the torque value based on the results of the feedback system) until the difference falls below the threshold value (which may indicate that the surgical drill torque is sufficient to drill through the bone). Similarly, once the surgical drill proceeds through the cortical portion of the bone, the difference between the anticipated sensor data and the actual sensor measurements may again exceed the threshold level (indicated, for example, that the torque of the surgical drill is too high for the trabecular portion of the bone). As such, the computing device 102 may reduce the torque (e.g., using feedback systems) of the surgical drill until the difference falls below the threshold value.

While the above provides an example of adjusting torque, additional or alternative control of the surgical drill (and more generally any surgical tool) may be implemented by the computing device 102. For instance, the computing device 102 may, when the difference exceeds the threshold value, may automatically stop the surgical maneuver (e.g., turn off the drilling). In such embodiments, the computing device 102 may restart the surgical maneuver (e.g., turn the drill on) after the difference falls below (or above) the threshold value and/or after the surgical plan is updated (e.g., after the surgical drill parameters are re-calibrated based on the most recent sensor measurements, based on the model estimation of the surgical drill parameters, combinations thereof, and/or the like). In some embodiments, the surgical maneuver may comprise the continued operation of the surgical drill autonomously or semi-autonomously. In such embodiments, the continued operation may be based on the difference remaining below (or above) the threshold value.

In some embodiments, the controlling may include registering one or more of the surgical tool and the surgical robot to the anatomical element. For instance, the difference between an anticipated operating parameter and an actual operating parameter may indicate that the surgical tool is operating in an incorrect location or is otherwise no longer properly aligned as specified in the surgical plan (e.g., drilling at an improper angle). In such embodiments, a registration 128 may be performed to register the surgical tool to the anatomical element (or vice versa). The registration 128 may utilize or implement one or more algorithms or models that identify the pose of the surgical tool, the robotic arm, and/or the anatomical element and register the coordinates associated therewith into a common coordinate space (e.g., a coordinate space of the surgical tool).

The present disclosure encompasses embodiments of the method 400 that comprise more or fewer steps than those described above, and/or one or more steps that are different than the steps described above.

As noted above, the present disclosure encompasses methods with fewer than all of the steps identified in FIGS. 3 and 4 (and the corresponding description of the methods 300 and 400), as well as methods that include additional steps beyond those identified in FIGS. 3 and 4 (and the corresponding description of the methods 300 and 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of estimating bone mineral density, the method comprising:
receiving one or more images of an anatomical element;
generating, based on the one or more images of the anatomical element, a three-dimensional mask for the anatomical element;
generating, based on the three-dimensional mask for the anatomical element, a transformed three-dimensional mask for at least a portion of the anatomical element;
filtering the one or more images of the anatomical element with the transformed three-dimensional mask for the at least a portion of the anatomical element; and
determining, based on the filtering, a bone mineral density for the at least a portion of the anatomical element.

2. The method of claim 1, wherein the anatomical element comprises a vertebra, and wherein the at least a portion of the anatomical element comprises a sub-anatomic region of the vertebra.

3. The method of claim 2, wherein the sub-anatomic region comprises a cortical region.

4. The method of claim 2, wherein the sub-anatomic region comprises a trabecular region.

5. The method of claim 2, wherein the transformed three-dimensional mask for the anatomical element comprises at least one of a trabecular mesh representing a trabecula portion of the vertebra or a cortical mask representing a cortical portion of the vertebra.

6. The method of claim 1, wherein generating the transformed three-dimensional mask for the at least a portion of the anatomical element comprises:
generating a trabecular mask for the anatomical element; and
generating a cortical mask for the anatomical element.

7. The method of claim 6, further comprising:
determining, for a trabecular portion of a vertebra and with the trabecular mask, the bone mineral density for the trabecular portion of the vertebra; and
determining, for a cortical portion of the vertebra and with the cortical mask, the bone mineral density for the cortical portion of the vertebra.

8. The method of claim 1, wherein the bone mineral density is measured based on Hounsfield Units.

9. The method of claim 1, further comprising:
determining an average bone mineral density for a plurality of sub-volumes of the anatomical element.

10. The method of claim 1, further comprising:
providing information describing the bone mineral density for the at least a portion of the anatomical element to a surgical robot as part of a surgical plan.

11. A method of operating a surgical robot, the method comprising:
receiving an estimate of bone mineral density for an anatomical element;
initiating a surgical maneuver on the anatomical element with the surgical robot;
receiving sensor information from a robotic sensor during the surgical maneuver;
comparing the sensor information with an anticipated sensor reading, wherein the anticipated sensor reading is determined based on the estimate of bone mineral density for the anatomical element; and
controlling the surgical robot during the surgical maneuver based on the comparison of the sensor information with the anticipated sensor reading.

12. The method of claim 11, wherein the surgical maneuver comprises at least one of drilling, milling, and cutting the anatomical element.

13. The method of claim 11, wherein the estimate of bone mineral density for the anatomical element is related to a particular volume of the anatomical element.

14. The method of claim 11, wherein the sensor information comprises at least one of drilling resistance, drilling torque, drilling depth, and force on an arm of the surgical robot.

15. The method of claim 11, wherein controlling the surgical robot comprises automatically stopping the surgical maneuver.

16. The method of claim 11, wherein controlling the surgical robot comprises allowing the surgical maneuver to continue in an autonomous fashion.

17. The method of claim 11, wherein the anatomical element comprises a vertebra.

18. The method of claim 11, wherein the estimate of bone mineral density for the anatomical element is determined with a CT scan or x-ray of the anatomical element.

19. The method of claim 11, wherein the estimate of bone mineral density for the anatomical element is determined preoperatively as a DXA t-score.

20. A system, comprising:
a first sensor;
a surgical tool;
a processor; and
a memory including data stored thereon that, when processed by the processor, cause the processor to:
receive an estimate of bone mineral density for an anatomical element;
initiate a surgical maneuver on the anatomical element with the surgical tool;
receive information from the first sensor during the surgical maneuver;
compare the information with an anticipated sensor reading, wherein the anticipated sensor reading is determined based on the estimate of bone mineral density for the anatomical element; and
control the surgical tool during the surgical maneuver based on the comparison of the information with the anticipated sensor reading.
